(19) United States
(12) Patent Application Publication
(10) Pub. No.: US 2025/0108193 A1
(43) Pub. Date: Apr. 3, 2025

(54) GUIDEWIRE DEVICE WITH CORE ALIGNMENT MECHANISM

(71) Applicant: SCIENTIA VASCULAR, INC., West Valley City, UT (US)

(72) Inventors: Adnan Rashid KHAN, Salt Lake City, UT (US); Edward J. SNYDER, Park City, UT (US)

(21) Appl. No.: 18/834,690

(22) PCT Filed: Feb. 8, 2023
(86) PCT No.: PCT/US2023/062171

§ 371 Date: Jul. 31, 2024

Related U.S. Application Data
(60) Provisional application No. 63/309,130, filed on Feb. 11, 2022.

(51) Int. Cl. A61M 25/09; A61M 25/01

(52) U.S. Cl. CPC A61M 25/09; A61M 25/0138; A61M 2025/09091; A61M 2025/09141; A61M 2025/09166

(57) ABSTRACT

Disclosed are guidewire devices, systems and methods of use, for example, in navigating tortuous environments like the human brain. The disclosed guidewires include a core that extends into an outer tube, where the distal section of the core may taper near its distal end. The guidewires may further include a washer having a slot to accommodate the distal core and maintain alignment of the distal core relative to the outer tube. When the outer tube is bent, the distal core will also bend such that the curvature of the distal core matches the curvature of the outer tube, due to the placement of the washer.

### CROSS-REFERENCE TO RELATED APPLICATIONS

This application claims priority to and the benefit of U.S. Provisional Patent Application No. 63/309,130, filed Feb. 11, 2022 and titled “Guidewire Device with Core Alignment Mechanism,” which is incorporated herein by reference in its entirety.

### BACKGROUND

### Technical Field

This disclosure relates to guidewire devices, systems and methods of use thereof. More specifically, this disclosure relates to guidewire devices comprising alignment mechanisms configured to orient a core wire and outer tube relative to each other.

### Related Technology

Guidewire devices are often used to lead or guide catheters or other interventional devices to a targeted anatomical location within a patient's body. Typically, guidewires are passed into and through a patient's vasculature in order to reach the target location, which may be at or near the patient's heart or neurovascular tissue, for example. Radiographic imaging is typically utilized to assist in navigating a guidewire to the targeted location. In many instances, a guidewire is left in place within the body during the interventional procedure where it can be used to guide multiple catheters or other interventional devices to the targeted anatomical location.

Some guidewire devices are constructed with a curved or bent tip to enable an operator to better navigate a patient's vasculature. With such guidewires, an operator can apply a torque to the proximal end of the guidewire or attached proximal handle in order to orient and point the tip in a desired direction. The operator may then direct the guidewire further within the patient's vasculature in the desired direction.

Tuning the flexibility of a guidewire device, particularly the distal sections of the guidewire device, is also a concern. In many circumstances, relatively high levels of flexibility are desirable in order to provide sufficient bendability of the guidewire to enable the guidewire to be angled through the tortuous bends and curves of a vasculature passageway to arrive at the targeted area. For example, directing a guidewire to portions of the neurovasculature requires passage of the guidewire through curved passages such as the carotid siphon and other tortuous paths.

Another concern related to guidewire devices is the ability of a given guidewire device to transmit torque from the proximal portion to the distal portion (i.e., the “torquability” of the guidewire device). As more of a guidewire is passed into and through a tortuous vasculature passageway, the amount of frictional surface contact between the guidewire and the vasculature increases, hindering easy movement of the guidewire through the vasculature passage. A guidewire with good torquability enables torsional forces at the proximal end to be transmitted through the guidewire to the distal end so that the guidewire can rotate and overcome the frictional forces.

Guidewires are available with various outer diameter sizes. Widely utilized sizes include 0.014, 0.016, 0.018, 0.024, and 0.035 inches, for example, though they may also be smaller or larger in diameter. Because torque transmission is a function of diameter, larger diameter guidewires typically have greater torque transmission (the ability to effectively transfer torque from proximal portions of the wire to more distal portions of the wire). On the other hand, smaller diameter guidewires typically have greater flexibility.

While such guidewire devices have provided many benefits, several limitations remain. For example, many of the design characteristics of a guidewire having a torque-transmitting tube, although functioning to provide increased torque transmission, work against and limit the shapeability of the guidewire tip.

### BRIEF SUMMARY

The present disclosure relates to intravascular devices such as guidewires having a core disposed within an outer tube and including one or more centering mechanisms configured to assist in radially positioning and/or centering the core within the outer tube. The one or more centering mechanisms beneficially reduce undesirable whip and/or snap movements of the guidewire (i.e., the centering mechanisms may improve rotational control), thereby enabling a user to have greater control and improved tactile handling of the guidewire. Further, such reduction in whip and/or snap movements beneficially reduces risks of tissue injury.

In one embodiment, a guidewire device includes a core having a proximal section and a tapered distal section. An outer tube is coupled to the core such that the tapered distal section of the core extends into and is surrounded by the outer tube. The outer tube may include a plurality of fenestrations to increase the flexibility of the outer tube. One or more centering mechanisms are positioned within at least a portion of the annular space between the core and the outer tube to assist in maintaining axial and/or radial alignment of the core within the tube.

In some embodiments, the core is formed from stainless steel, and the tube is formed from a super-elastic material such as nitinol, though other suitable biocompatible materials may additionally or alternatively be used. Some embodiments further include a marker coil positioned between an outer surface of a distal section of the core and an inner surface of the outer tube. The marker coil may be formed from a radiopaque material.

In some embodiments, a centering mechanism includes one or more centering washers disposed within the annular space. The centering washer may be formed from a suitable metal or metal alloy, such as nitinol or stainless steel, or alternatively may be formed from a suitable polymer as described below. A centering washer as used herein is sized to fit within and fill a portion of the annular space. That is, the centering washer has an outer diameter equal to or less than an inner diameter of the tube and has an inner aperture sized to be greater than the coincident portion of the core.

Alternatively, some embodiments include an offset washer that functions to intentionally offset the distal section of the core from the longitudinal axis of the device. As described in greater detail below, an offset washer enables greater plastic deformation of the core at or near the offset portion, which is beneficial in applications where a shaped/bent tip is desired.

Additional features and advantages will be set forth in part in the description that follows, and in part will be obvious from the description, or may be learned by practice of the embodiments disclosed herein. The objects and advantages of the embodiments disclosed herein will be realized and attained by means of the elements and combinations particularly pointed out in the appended claims. It is to be understood that both the foregoing brief summary and the following detailed description are exemplary and explanatory only and are not restrictive of the embodiments disclosed herein or as claimed.

### BRIEF DESCRIPTION OF THE DRAWINGS

A more particular description of the invention briefly described above will be rendered by reference to specific embodiments which are illustrated in the appended drawings. Understanding that these drawings depict only typical embodiments of the invention and are not limiting of its scope, the invention will be described and explained with additional specificity and detail through the use of the accompanying drawings in which:

FIG. 1 illustrates an embodiment of a guidewire device having a core and an outer tube and which may utilize one or more of the core centering mechanisms described herein;

FIGS. 2A and 2B illustrate a cross section of a vessel in which a guidewire has been inserted, with FIG. 2A showing a straight section of the vessel and FIG. 2B showing a curved section of the vessel where the core tends to move out of axial alignment with the outer tube;

FIG. 3 illustrates another view of the guidewire device of FIG. 1 showing certain internal features;

FIGS. 4A-4F illustrate embodiments of a tube structure including a centering washer or an offset washer;

FIGS. 5A and 5B illustrate an embodiment of a guidewire device including a centering washer; and

FIGS. 5C and 5D illustrate an embodiment of a guidewire device including an offset washer.

### DETAILED DESCRIPTION

### Overview

Some guidewires are constructed with a core and an outer member or tube that surrounds the distal portion of the core. The core is often formed of a relatively stiff material such as stainless steel while the outer member is often formed from a more flexible material such as nitinol. The outer member may also include machined transverse slots to increase flexibility. The intent behind such designs is to reduce the diameter of the core in the distal sections of the guidewire in order to increase the flexibility of the wire, while utilizing the larger outer diameter of the outer member for effective torque transmission. Because torsional forces are primarily transmitted through the outer sections of a cross-section of a member, the tube is configured to provide a path for increased transmission of torque as compared to torque through the core alone. While such guidewires have seen success, several limitations remain. In particular, the difference in diameter between the outer tube and the core creates an annular space within the coincident sections of the guidewire. Because the outer tube is by design more elastic than the core wire, as the guidewire navigates bends in the vasculature, the core will often be positioned off-center relative to the center line of the outer tube.

This off-centering can disrupt the smooth distal transmission of rotational and/or axial movement, causing a buildup and sudden release of forces which lead the guidewire to move with a “snap” and/or “whip” to a preferential rotational location. This disruption to the tactile feel and control of the guidewire can make it more difficult for the operator to rotationally position the guidewire as intended, raising the risk of interventional procedure delays, suboptimal outcomes, inability to access the target location, or even tissue injury.

Embodiments described herein can beneficially address one or more of the foregoing limitations of conventional guidewire devices by including one or more alignment washers configured to limit movement of the core relative to the outer tube. Such alignment washers, and devices in which such alignment washers can be included, are described in more detail below.

### Example Guidewire Structural Features

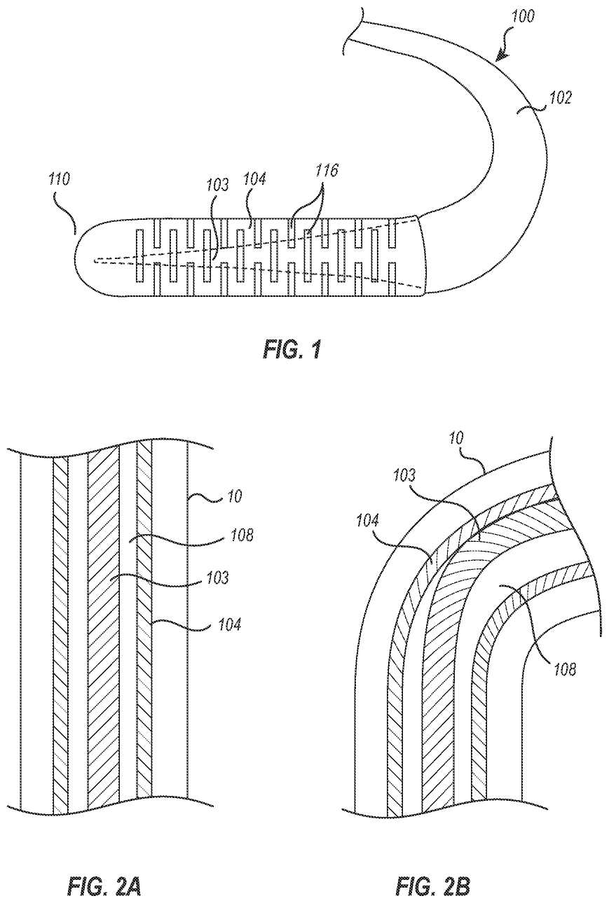

FIG. 1 schematically illustrates a guidewire device 100 suitable for utilizing one or more features of the present disclosure. The illustrated guidewire 100 includes a core 102 and an outer tube 104. The core 102 includes a distal section 103 that extends into the outer tube 104 as shown. The distal core 103 may be tapered, either continuously or in one or more discrete sections, so that the distal section 103 has a smaller diameter and greater flexibility than more proximal sections of the core 102. For example, the distal section 103 may be ground so as to progressively taper to a smaller diameter at the distal end. In some embodiments, the distal section 103 may be flattened into a ribbon-like shape with a flat, rectangular, or oblong cross section.

The core 102 and the tube 104 are typically formed from different materials. For example, the tube 104 is preferably formed from a relatively flexible and elastic material such as nitinol, whereas the core 102 may be formed from a relatively less flexible and elastic material such as stainless steel. Forming the core 102 from stainless steel may be advantageous because it allows the distal tip to hold a shape when selectively bent/shaped by an operator (i.e., it is capable of manual elastic deformation) and because stainless steel provides sufficient modulus of elasticity to provide more responsive translational movement. While these materials are presently preferred, other suitable materials such as polymers or other metals/alloys may additionally or alternatively be utilized.

In the illustrated device, the outer diameter of the core 102 and the inner diameter of the tube 104 are substantially similar at the attachment point where the core 102 enters the tube 104. In some embodiments, the core 102 outer diameter and the tube 104 inner diameter have different diameters at the attachment point, with the difference in diameter being compensated for by a weld, solder, adhesive, or other means of structural attachment, or by positioning a portion of a centering mechanism (e.g., centering coil, braid, collet, bushing, tube, and/or other bushing structure) at the attachment point.

The tube 104 is coupled to the core 102 (e.g., using adhesive, soldering, and/or welding) in a manner that beneficially allows torsional forces to be transmitted from the core 102 to the tube 104 and thereby to be further transmitted distally by the tube 104. A medical grade adhesive or other suitable material may be used to couple the tube 104 to the core wire 102 at the distal end 110 of the device. This adhesive may also form an atraumatic covering at the distal end 110 of the device.

The outer tube 104 may include a cut pattern that forms fenestrations 116 in the tube. The pattern of fenestrations 116 may be arranged to provide desired flexibility characteristics to the tube 104, including the promotion of preferred bending directions, the reduction or elimination of preferred bending directions, or gradient increases in flexibility along the longitudinal axis, for example. Examples of cut patterns and other guidewire device features that may be utilized in the guidewire devices described herein are provided in detail in U.S. Pat. Nos. 10,821,268, 11,052,228, and in U.S. Pat. No. 11,369,351, the entireties of each of which are incorporated herein by this reference.

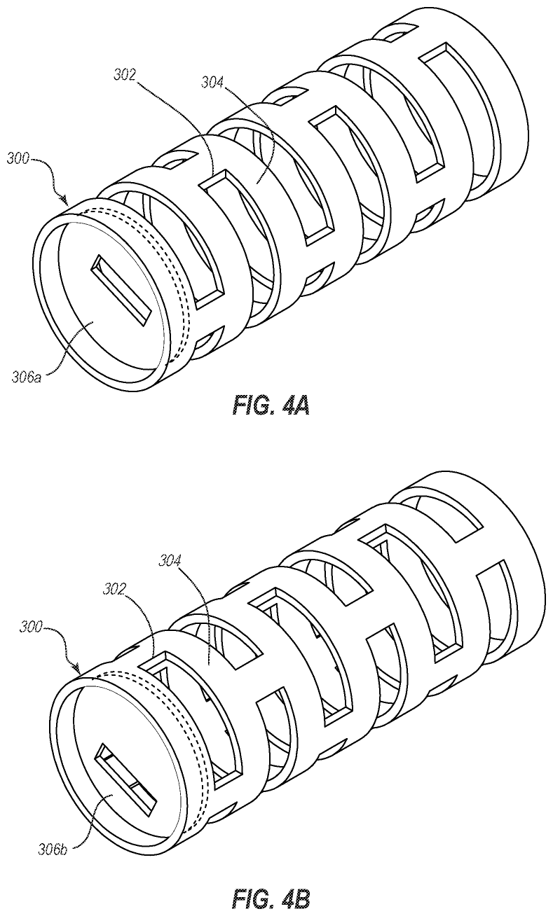

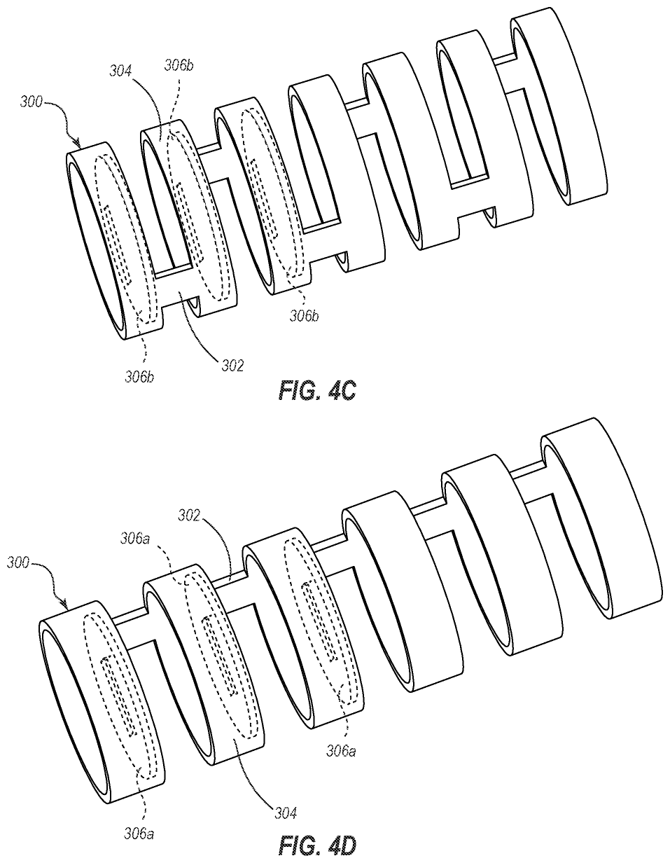

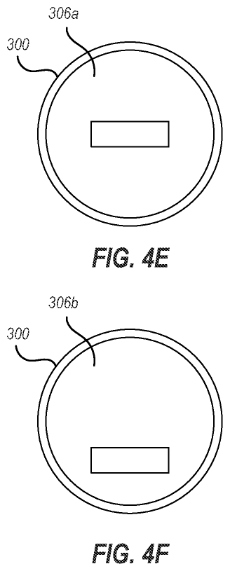

Particular examples are also shown in the outer tubes (i.e., tube structures) of FIGS. 4A-4D. Suitable cut patterns include, for example, a three-beam cut pattern, two-beam cut pattern, and one-beam cut pattern. A “two-beam” cut pattern or section refers to a section of the outer tube comprising two axially extending “beams” between each pair of consecutive circumferentially extending “rings,” a “one-beam” cut pattern or section refers to a section of the outer tube comprising a single axially extending “beam” between each pair of consecutive circumferentially extending “rings,” and so on.

The beams and rings may be arranged along the length of a given section of the tube to form various cut patterns, including a linear pattern, helical pattern, or a distributed pattern that is non-helical and non-linear (see U.S. Pat. No. 11,369,351).

The proximal section of the guidewire device 100 (the portion extending proximally from the tube 104) extends proximally to a length necessary to provide sufficient guidewire length for delivery to a targeted anatomical area. The guidewire device 100 typically has a length ranging from about 50 cm to about 350 cm depending on particular application needs. The tube 104 may have a length ranging from about 20 cm to about 65 cm, more typically about 30 cm to about 55 cm, such as about 35 cm to about 45 cm, though other tube lengths may also be utilized according to particular application needs.

The guidewire device 100 may have a diameter of about 0.014 inches to about 0.035 inches, though larger or smaller sizes may also be utilized depending on particular application needs, and the features of the present disclosure are not necessarily limited to certain guidewire sizes. Some embodiments may have outer diameter sizes corresponding to standard guidewire sizes such as 0.014 inches, 0.016 inches, 0.018 inches, 0.024 inches, 0.035 inches, or other such sizes common to guidewire devices.

The distal section 103 of the core 102 may taper to a diameter of about 0.002 inches, or a diameter within a range of about 0.001 to 0.005 inches. In some embodiments, the distal tip of the core may be flattened (e.g., to a rectangular cross section) to further enhance bending flexibility while minimizing reductions in cross-sectional area needed for tensile strength. In such embodiments, the cross section may have dimensions of about 0.001 inches by 0.003 inches, for example.

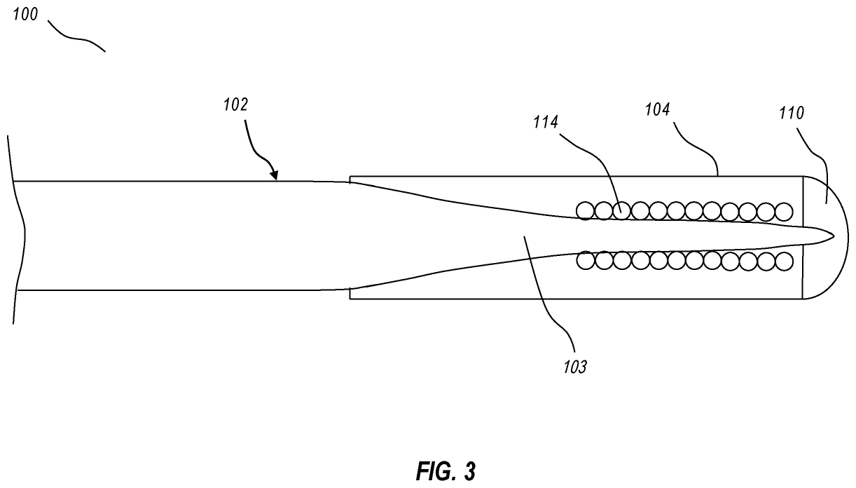

FIG. 3 illustrates an additional view of the guidewire device 100 of FIG. 1 shown in cross-section. As shown, the guidewire device can include a marker coil 114 positioned upon at least a portion of the distal section 103 of the core 102. The marker coil 114 is preferably formed from one or more radiopaque materials, such as platinum group, gold, silver, palladium, iridium, osmium, tantalum, tungsten, bismuth, dysprosium, gadolinium, and the like. In the illustrated embodiment, the marker coil 114 is disposed at or near the distal end of the device. In some embodiments, the marker coil 114 has a length that substantially coincides with the length of the tube 104. In other embodiments, the marker coil 114 is shorter. For example, the marker coil 114 may have a length of 1, 2, 4, 6, 8, 10, or 12 cm, or may have a length within a range defined by any two of the foregoing values.

Other coils may additionally or alternatively be included in the guidewire device 100. Such coils may be utilized to, for example, enhance shapeability of the distal tip of the device and/or further assist in aligning the core relative to the outer tube. Additional disclosure related to such coil arrangements suitable for use with the presently disclosed guidewire devices is provided in U.S. Pat. No. 10,953,202, United States Patent Application Pub. No. 2020/0222672 and in United States Patent Application Pub. No. 2021/0228845, each of which is incorporated herein by reference in its entirety.

Such coils may be radiopaque or non-radiopaque. The term “non-radiopaque” refers to materials that are not typically utilized as for marking/visualization purposes in the field of intravascular devices, even if such a material technically has some level of radiopacity. Stainless steel is an example of a “non-radiopaque” material.

### Core and Tube Misalignment

FIGS. 2A and 2B show a cross-sectional view of a vessel 10 in which the distal portion of a guidewire device 100 has been inserted. As shown in FIG. 2A, in relatively straighter sections of the vessel 10, the outer tube 104 and the distal core 103 may be essentially radially centered. An annular space 108 exists within the tube 104 between the inner surface of the tube 104 and the outer surface of the distal core 103.

FIG. 2B shows a curved section of the vessel 10 illustrating one example of how the distal core 103 may become off-center relative to the outer tube 104. As the guidewire curves to match the curvature of the vessel 10, the relatively less flexible distal core 103 will not bend as readily as the coincident section of the outer tube 104. This causes the distal core 103 to move off center within the tube 104. This misalignment can disrupt smooth rotational and/or axial movement, causing a buildup and sudden release of energy which leads the guidewire to undesirably “snap” and/or “whip”.

Issues associated with off-centered alignment of the core may be more problematic as guidewire size goes up. As guidewire size goes up, the inner diameter of the tube 104 may increase more than the outer diameter of the distal core 103. That is, the diameter of the tube 104 may be increased to give greater torquability but the diameter of the distal core 103 may be substantially maintained to preserve the flexibility profile of the distal portion of the device. This means that larger sized guidewires will often have larger annular spaces 108 and are therefore more prone to misalignment of the core relative to the longitudinal axis of the tube 104.

By way of example, where a 0.014 inch guidewire device may in some circumstances function adequately without any core centering mechanism, a larger sized guidewire such as a 0.018 or 0.024 inch guidewire may increasingly exhibit problems associated with core off-centered alignment. In other circumstances, however, the core centering mechanisms described herein may be beneficially used even in smaller guidewires such as a 0.014 inch guidewire.

### Example Alignment Mechanisms

FIGS. 4A-4D illustrate various embodiments of a tube structure including an alignment mechanism in the form of one or more centering washers 306a and/or one or more offset washers 306b. The tube structures 300 are illustrated with a variety of fenestrations or cut patterns, resulting in various configurations of beams 302 and rings 304. The tube structures 300 can correspond to tube structure 104 described in relation to FIGS. 1-3.

The washers 306a, 306b (generically referred to as “washers 306”) can be sized such that an outer diameter of the washer 306 fits inside, and is substantially equal to, an inner diameter of the tube structure 300. One or more centering washers 306a and/or one or more offset washers 306b may be included in any of the guidewire devices described herein.

The illustrated washers 306 include an aperture for receiving the distal section 103 of the core 102. In embodiments where the washer 306 is disposed coincident with a flattened portion of the core 102, the aperture is formed as a slot that substantially corresponds to the cross-sectional shape of the flattened core 102. That is, the slot of the washer 306 can be sized such that an opening of the slot is larger or slightly larger than the size of the distal section 103 of the core 102, thereby allowing the distal section 103 to extend through the slot. For example, the slot of the washer 306 may be sized to allow the distal section 103 of the core 102 having a size within a range of about 0.001 to 0.005 inches to pass through and extend past the slot of the washer 306.

The washer 306 can thereby radially anchor the radial position of the distal section 103 of the core 102 relative to the tube structure 300. This beneficially assists in centering the core 102 with respect to the tube 104 and can thereby reduce undesirable snap and/or whip effects during use of the device. Radially anchoring the distal section 103 of the core 102 enables a curvature of the distal section 103 of the core 102 to better match a curvature of the tube structure 300 during use of the device.

In some embodiments, one or more washers 306 are substantially circular. In some embodiments, one or more washers 306 are non-circular, such as substantially oval shaped or comprising a spoke and hub structure.

As shown in FIG. 4A, the centering washer 306a has a slot substantially centered at the radial center of the centering washer 306a. As shown in FIG. 4B, the offset washer 306b includes a slot intentionally offset from the radial center of the washer 306b, such as positioned just below a longitudinal axis of the washer 306b. Other embodiments may position the slot just above the longitudinal axis of the washer 306b or in any other position on the washer 306b.

Guidewire devices may have one washer 306 for maintaining radial alignment of the core 102 in a guidewire device 100. Alternatively, guidewire devices may have more than one washer 306. For example, FIG. 4C illustrates an embodiment with a plurality of offset washers 306b, and FIG. 4D illustrates an embodiment with a plurality of centering washers 306a.

FIGS. 4E and 4F illustrate a front facing view of a tube structure 300 including a centering washer 306a (FIG. 4E) or an offset washer 306b (FIG. 4F). As illustrated in FIG. 4E, the centering washer 306a has a slot that is substantially centered in the radial center of the centering washer 306a (i.e., coincident with a central longitudinal axis passing through the tube 300 and the washer 306a). As illustrated in FIG. 4F, the offset washer 306b has a slot that is offset from a central longitudinal axis of the washer 306b.

Use of Guidewire Device with Alignment Mechanism

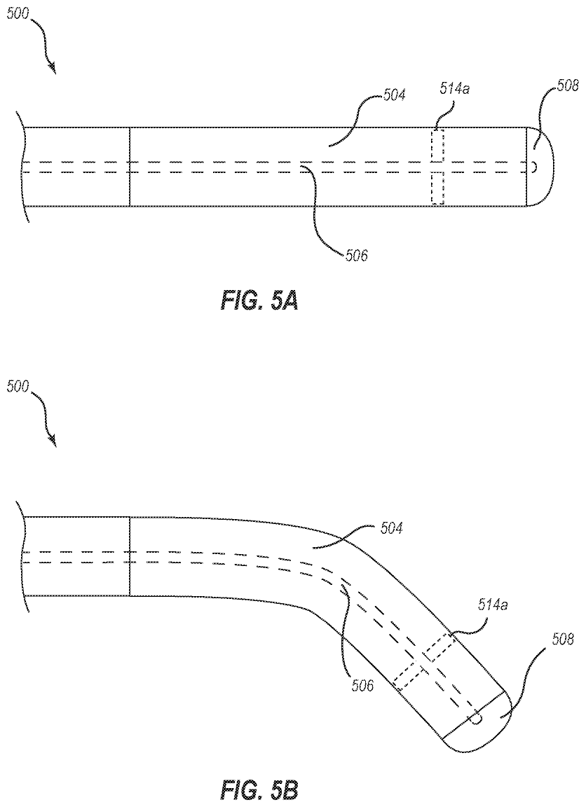

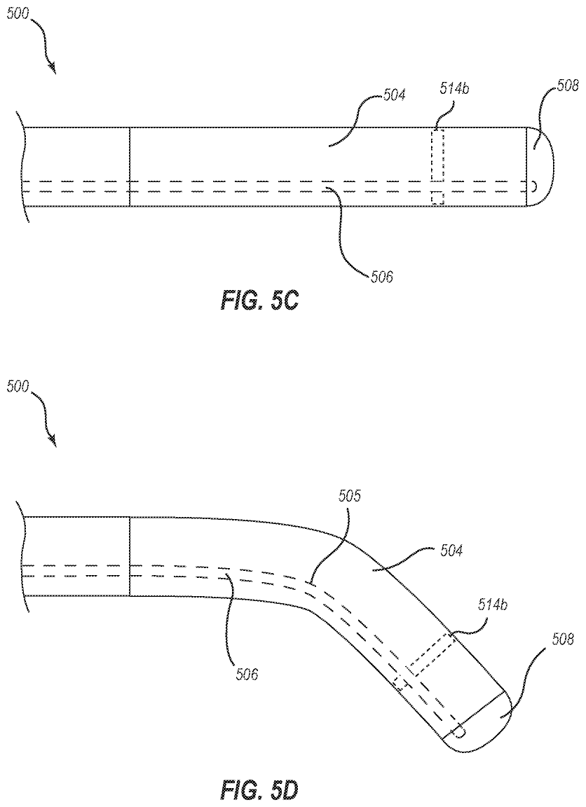

FIGS. 5A and 5B illustrate a distal region of an exemplary guidewire device 500, which includes features similar to the other guidewire devices disclosed herein. Any of the guidewire devices described in FIGS. 1-4F may be taken to correspond to guidewire device 500, and any of the centering or offset washers described in FIGS. 4A-4F may be taken to respectively correspond to washers centering washers 514a and offset washers 514b.

The device 500 includes a tube 504, a core 506, and a distal tip 508. In this embodiment, the core 506 extends through a centering washer 514a. The centering washer 514a maintains radial alignment of the core 506 relative to the tube 504. The centering washer 514a may be attached to the core 506 and/or tube 504 via an adhesive, soldering, welding, or other suitable attachment means.

As shown, the centering washer 514a beneficially keeps the core 506 in radial alignment with the tube 504 during use of the guidewire device 500. In contrast, in the absence of a centering washer 514a, the core 506 can more readily become misaligned from the tube 504 during bending. The centering washer 514a thus beneficially assists in aligning the curvature of the core 506 with the curvature of the tube 504. When a curvature is formed in the tube 504, the centering washer 114a functions to impart the same curvature to the core 506. In contrast, a guidewire device omitting a centering mechanism, when curved at the tube 504, may instead extend until abutting against the inner surface of the tube 504 before being forced to curve (e.g., as illustrated in FIGS. 2A and 2B).

As illustrated in FIGS. 5C and 5D, the offset washer 514b intentionally offsets the core 506 relative to the center of the tube 504 and thereby positions the core 506 closer to one side of the tube 504.

One advantage of a guidewire device comprising one or more offset washers 514b involves better enabling the formation of a shaped/bent distal tip. For example, when the distal end of the device 500 is bent/shaped in the same direction of the offset, the core 506 is forced through a smaller radius of curvature at bending point 505 than if the core 506 were centered. This intentional offsetting of the core 506 thus increases the plastic deformation of the core 506 at the bending point 505. This increased plastic deformation of the core 506 beneficially allows for better maintenance of the bent shape. That is, the shaped distal tip of the device is less likely to lose its bent shape, which often occurs when a bent tube 504 (which is often superelastic) biases toward the straight position and thus imparts straightening forces against the core 506.

Embodiments with one or more offset washers 514b can thus beneficially enable the distal tip of the guidewire device to be shaped to a desired position and to remain in the shaped position for a sufficiently extended period of time. With conventional guidewire devices, problems related to shapeability often occur as a result of a mismatch in properties between the tube structure and the internal components (the core and coil). Tube structures are typically formed from nitinol or other super-elastic materials. Such tubes will be, upon being bent or shaped, biased toward their original (straight) position, and will thereby impart recovery forces against any shapeable internal components, resulting in deformation and a loss of the customized shape of the tip. The enhanced plastic deformation of the core 506 enabled by the offset washer 514b enhances the ability to resist such recovery forces and thereby promote maintenance of the shaped tip.

Often, for example, a conventional guidewire will have a shaped tip prior to deployment, but the shaped tip will be lost or degraded during use of the guidewire as the super-elastic tube flexes toward its original shape in opposition to the desired tip shape. The recovery forces imparted by the tube thus act against the internal components to reduce or degrade the desired shape set by the user. In contrast, the embodiments described herein that include one or more offset washers have enhanced resistance to the forces that tend to disrupt the custom shape of the guidewire tip.

### Additional Terms & Definitions

The terms “approximately,” “about,” and “substantially” as used herein represent an amount or condition close to the stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a stated amount or condition.

Unless otherwise indicated, numbers expressing quantities, proportions, percentages, or other measurements used in the specification and claims are to be understood as optionally being modified by the term “about” or its synonyms, even if the term does not expressly appear. Any numerical range recited herein is intended to include all subranges subsumed therein. When ranges are given, any endpoints of those ranges and/or numbers within those ranges can be combined within the scope of the present disclosure. Plural use of terms encompasses singular use of the terms and vice versa.

The intravascular devices (e.g., guidewire devices) disclosed herein should be understood as comprising/including disclosed components, and may therefore include additional components not specifically described. Any of the individual components of the intravascular device may optionally be omitted.

The intravascular devices disclosed herein are, optionally, essentially free or completely free of components that are not specifically described. That is, non-disclosed components may optionally be omitted or essentially omitted from the disclosed intravascular devices. For example, a cutting pattern for the outer tube, a coil arrangement, or core joint feature that is not specifically described as being included in the disclosed intravascular device may be optionally excluded (i.e., essentially omitted or completely omitted).

Any headings and subheadings used herein are for organizational purposes only and are not meant to be used to limit the scope of the description or the claims.

### EXAMPLE EMBODIMENTS

The disclosed intravascular device may include, but is not necessarily limited to, the features recited by the following embodiments:

Embodiment 1: an intravascular device, comprises: a core having a proximal section and a distal section; an outer tube coupled to the core such that the distal section of the core passes into and is encompassed by the outer tube, the outer tube and the core defining an annular space between an inner surface of the outer tube and an outer surface of the core; and an alignment washer disposed in the annular space, the alignment washer including a slot through which the distal section of the core passes.

Embodiment 2: The device of Embodiment 1, wherein the alignment washer is a centering washer, wherein the slot is substantially centered in the centering washer such that the portion of the core passing through the centering washer is maintained in a substantially radially centered position relative to the outer tube.

Embodiment 3: The device of any one of Embodiments 1-2, wherein the alignment washer is an offset washer, wherein the slot is offset from the radial center of the offset washer such that the portion of the core passing through the offset washer is maintained closer to one side of the outer tube than an opposing side.

Embodiment 4: The device of any one of Embodiments 1-3, further comprising a radiopaque marker coil.

Embodiment 5: The device of any one of Embodiments 1-4, wherein the radiopaque marker coil is disposed around the distal section of the core and is encompassed by the outer tube.

Embodiment 6: The device of any one of Embodiments 1-5, further comprising one or more alignment coils.

Embodiment 7: The device of Embodiment 6, wherein the one or more alignment coils are non-radiopaque.

Embodiment 8: The device of any one of Embodiments 1-7, wherein the slot of the alignment washer has a rectangular shape.

Embodiment 9: The device of any one of Embodiments 1-8, wherein the portion of the core passing through the slot has a rectangular cross-sectional shape.

Embodiment 10: The device of any one of Embodiments 1-9, wherein the outer tube includes a cut pattern which forms a plurality of axially extending beams coupling a plurality of circumferentially extending rings.

Embodiment 11: The device of any one of Embodiments 1-10, wherein the outer tube comprises one or more of a three-beam section, two-beam section, or one-beam section.

Embodiment 12: The device of Embodiment 10, wherein the beams and rings of the outer tube are arranged to form one or more of a linear pattern, helical pattern, or a distributed pattern that is non-helical and non-linear.

Embodiment 13: The device of any one of Embodiments 1-12, wherein at least a portion of the distal section of the core comprises a flat ribbon.

Embodiment 14: The device of any one of Embodiments 1-13, wherein the outer tube comprises a superelastic material.

Embodiment 15: The device of Embodiment 14, wherein the outer tube comprises nitinol.

Embodiment 16: The device of any one of Embodiments 1-15, wherein the core comprises stainless steel.

Embodiment 17: The device of any one of Embodiments 1-16, wherein the intravascular device is a guidewire.

Embodiment 18: The device of any one of Embodiments 1-17, wherein the device comprises multiple alignment washers.

1. An intravascular device, comprising:
a core having a proximal section and a distal section;
an outer tube coupled to the core such that the distal section of the core passes into and is encompassed by the outer tube, the outer tube and the core defining an annular space between an inner surface of the outer tube and an outer surface of the core; and
an alignment washer disposed in the annular space, the alignment washer including a slot through which the distal section of the core passes.

2. The device of claim 1, wherein the alignment washer is a centering washer, wherein the slot is substantially centered in the centering washer such that the portion of the core passing through the centering washer is maintained in a substantially radially centered position relative to the outer tube.

3. The device of claim 1, wherein the alignment washer is an offset washer, wherein the slot is offset from the radial center of the offset washer such that the portion of the core passing through the offset washer is maintained closer to one side of the outer tube than an opposing side.

4. The device of claim 1, further comprising a radiopaque marker coil.

5. The device of claim 4, wherein the radiopaque marker coil is disposed around the distal section of the core and is encompassed by the outer tube.

6. The device of claim 1, further comprising one or more alignment coils.

7. The device of claim 6, wherein the one or more alignment coils are non-radiopaque.

8. The device of claim 1, wherein the slot of the alignment washer has a rectangular shape.

9. The device of claim 8, wherein the portion of the core passing through the slot has a rectangular cross-sectional shape.

10. The device of claim 1, wherein the outer tube includes a cut pattern which forms a plurality of axially extending beams coupling a plurality of circumferentially extending rings.

11. The device of claim 10, wherein the outer tube comprises one or more of a three-beam section, two-beam section, or one-beam section.

12. The device of claim 10, wherein the beams and rings of the outer tube are arranged to form one or more of a linear pattern, helical pattern, or a distributed pattern that is non-helical and non-linear.

13. The device of claim 1, wherein at least a portion of the distal section of the core comprises a flat ribbon.

14. The device of claim 1, wherein the outer tube comprises a superelastic material.

15. The device of claim 14, wherein the outer tube comprises nitinol.

16. The device of claim 1, wherein the core comprises stainless steel.

17. The device of claim 1, wherein the intravascular device is a guidewire.

18. The device of claim 1, wherein the device comprises multiple alignment washers.

19. An intravascular device, comprising:
a core having a proximal section and a distal section;
an outer tube coupled to the core such that the distal section of the core passes into and is encompassed by the outer tube, the outer tube and the core defining an annular space between an inner surface of the outer tube and an outer surface of the core; and
a centering washer disposed in the annular space, the centering washer including a slot through which the distal section of the core passes,
wherein the slot is substantially centered in the centering washer such that the portion of the core passing through the centering washer is maintained in a substantially radially centered position relative to the outer tube, and
wherein the slot of the centering washer has a rectangular shape and the portion of the core passing through the slot has a corresponding rectangular cross-sectional shape.

20. An intravascular device, comprising:
a core having a proximal section and a distal section;
an outer tube coupled to the core such that the distal section of the core passes into and is encompassed by the outer tube, the outer tube and the core defining an annular space between an inner surface of the outer tube and an outer surface of the core; and
an offset washer disposed in the annular space, the offset washer including a slot through which the distal section of the core passes,
wherein the slot is offset from the radial center of the offset washer such that the portion of the core passing through the offset washer is maintained closer to one side of the outer tube than an opposing side, and
wherein the slot of the centering washer has a rectangular shape and the portion of the core passing through the slot has a corresponding rectangular cross-sectional shape.
